(19) United States
(12) Patent Application Publication
(10) Pub. No.: US 2021/0237077 A1
(43) Pub. Date: Aug. 5, 2021

(54) APPARATUS FOR PLATELET FUNCTION TEST USING SPECKLE DECORRELATION TIME ANALYSIS

(71) Applicant: GIST(Gwangju Institute of Science and Technology), Gwangju (KR)

(72) Inventors: Eui Heon CHUNG, Gwangju (KR); Hee Jae JEON, Gwangju (KR); Muhammad Mohsin QURESHI, Gwangju (KR); Seung Yeob LEE, Gwangju (KR)

(73) Assignee: GIST(Gwangju Institute of Science and Technology), Gwangju (KR)

(21) Appl. No.: 17/038,447

(22) Filed: Sep. 30, 2020

(30) Foreign Application Priority Data
Feb. 3, 2020 (KR) 10-2020-0012646

(51) Int. Cl. B01L 3/00; G01N 33/49

(52) U.S. Cl. CPC B01L 3/502761; G01N 33/4915; B01L 2200/0668; B01L 2400/049; B01L 2300/0864; B01L 2300/0867; B01L 2300/0883; B01L 2300/0816

(57) ABSTRACT

The present invention relates to an apparatus for a platelet function test using speckle decorrelation time analysis. An apparatus for a platelet function test according to an exemplary embodiment of the present invention may include: a laser unit for irradiating a blood sample including platelets with a laser; an image capturing unit for obtaining a speckle image of the blood sample irradiated with the laser; and a control unit for determining the speckle decorrelation time for platelets.

### CROSS-REFERENCE TO RELATED APPLICATION

This application claims priority to and the benefit of Korean Patent Application No. 10-2020-0012646, filed on Feb. 3, 2020, the disclosure of which is incorporated herein by reference in its entirety.

### BACKGROUND

### 1. Field of the Invention

The present invention relates to an apparatus for a platelet function test, and more particularly, to an apparatus for a platelet function test using speckle decorrelation time analysis.

### 2. Discussion of Related Art

A platelet function test is often used as a screening test of a congenital platelet dysfunction or before surgery, and is an important test especially for discriminating a hemorrhagic disease caused by congenital or acquired platelet dysfunction in which abnormal platelet counts are absent.

Recently, such platelet function tests are often used even for tests for an increase in bleeding tendency or for drug resistance due to antiplatelet drugs used for the treatment and prevention of cardiovascular diseases.

The bleeding time (BT) test is a bleeding time measurement test and has been used as a platelet function screening test to date. However, there are problems in that currently used platelet function tests are difficult to standardize, have little clinical utility, and require the use of invasive methods, and accordingly, there is a need for an objective measurement method capable of measuring platelet function.

The conventional techniques devised to solve the above-mentioned problems have a disadvantage in that the test cost is increased because a two-step test is required for the functional test of platelets.

### PRIOR ART DOCUMENT

[Patent Document]
(Patent Document 1) [Patent Document 1] Korean Patent No. 10-1749796

### SUMMARY OF THE INVENTION

The present invention has been created in order to solve the above-described problems, and an object of the present invention is to provide an apparatus for a platelet function test using speckle decorrelation time analysis.

Further, an object of the present invention is to provide an apparatus for a platelet function test for determining the speckle decorrelation time for platelets by analyzing speckle images obtained from a blood sample irradiated with a laser.

The objects of the present invention are not limited to the above-mentioned objects, and other objects that are not mentioned may be clearly understood by those skilled in the art from the following description.

To achieve the aforementioned objects, an apparatus for a platelet function test according to an exemplary embodiment of the present invention may include: a laser unit for irradiating a blood sample including platelets with a laser; an image capturing unit for obtaining a speckle image of the blood sample irradiated with the laser; and a control unit for determining the speckle decorrelation time for platelets.

In an exemplary embodiment, the apparatus for a platelet function test further includes a microfluidic chip for moving the blood sample, in which the laser unit may irradiate the blood sample moving through the microfluidic chip with a laser.

In an exemplary embodiment, the microfluidic chip may include an injection unit into which the blood sample is injected, a movement channel through which the injected blood sample moves, and a discharge unit through which the moved blood sample is discharged.

In an exemplary embodiment, the apparatus for a platelet function test may further include a vacuum unit which is coupled to the microfluidic chip to move the blood sample by vacuum pressure.

In an exemplary embodiment, the vacuum unit may include a movement tube through which the discharged blood sample moves, a first valve which is coupled to the movement tube to open and close a flow of the blood sample, and a vacuum pump which is coupled to the movement tube to form a vacuum state inside the movement tube according to the opening and closing of the first valve.

In an exemplary embodiment the vacuum unit may further include a buffer unit which is coupled to the movement tube to buffer the movement of the blood sample.

In an exemplary embodiment, the vacuum pump may form a vacuum state inside the movement tube after the first valve is closed, the first valve may be opened after the vacuum state inside the movement tube is formed, and the movement tube may move the blood sample based on vacuum pressure generated as the first valve is opened.

In an exemplary embodiment, the control unit may measure the function of platelets based on the speckle decorrelation time for platelets.

Specific details for achieving the aforementioned objects will become apparent with reference to the exemplary embodiments to be described below in detail together with the accompanying drawings.

However, the present invention is not limited to the exemplary embodiments to be disclosed below and may be implemented in various other forms, and the present invention is provided for rendering the disclosure of the present invention complete and for fully indicating the scope of the present invention to those with ordinary skill in the art (hereinafter, “those skilled in the art”) to which the present invention pertains.

### BRIEF DESCRIPTION OF THE DRAWINGS

The above and other objects, features and advantages of the present invention will become more apparent to those of ordinary skill in the art by describing in detail exemplary embodiments thereof with reference to the accompanying drawings, in which:

FIG. 1A is a view illustrating the functional configuration of an apparatus for a platelet function test according to an exemplary embodiment of the present invention;

FIG. 1B is a view illustrating a microfluidic chip according to an exemplary embodiment of the present invention;

FIG. 2 is a view illustrating a set of speckle images according to an exemplary embodiment of the present invention;

FIG. 3A is a view illustrating a speckle decorrelation time graph for flow rate according to an exemplary embodiment of the present invention;

FIG. 3B is a view illustrating a speckle decorrelation time graph for particle size according to an exemplary embodiment of the present invention;

FIG. 3C is a view illustrating a speckle decorrelation time graph for particle concentration according to an exemplary embodiment of the present invention;

FIG. 4 is a view illustrating a speckle decorrelation time graph for the presence and absence of platelets according to an exemplary embodiment of the present invention;

FIGS. 5A and 5B are views illustrating a speckle decorrelation time graph for the presence and absence of platelets according to an exemplary embodiment of the present invention;

FIG. 6A to 6E are views illustrating a speckle decorrelation time graph for various platelet activation materials according to an exemplary embodiment of the present invention; and

FIG. 7A to 7D are views illustrating a light transmittance graph according to an exemplary embodiment of the present invention.

### DETAILED DESCRIPTION OF EXEMPLARY EMBODIMENTS

Since the present invention may be modified into various forms and include various exemplary embodiments, specific exemplary embodiments will be illustrated in the drawings and described in detail.

Various features of the invention disclosed in the claims can be better understood in view of the drawings and the detailed description. Apparatuses, methods, manufacturing methods, and various embodiments disclosed herein are provided for the purpose of illustration. The disclosed structural and functional features are intended to enable those skilled in the art to specifically practice various embodiments, and are not intended to limit the scope of the invention. The disclosed terms and sentences are intended to explain various features of the disclosed invention in an easy-to-understand manner and are not intended to limit the scope of the invention.

In the description of the present invention, when it is determined that the detailed description for the related known technology can unnecessarily obscure the gist of the present invention, the detailed description thereof will be omitted.

Hereinafter, an apparatus for a platelet function test using speckle decorrelation time analysis according to an exemplary embodiment of the present invention will be described.

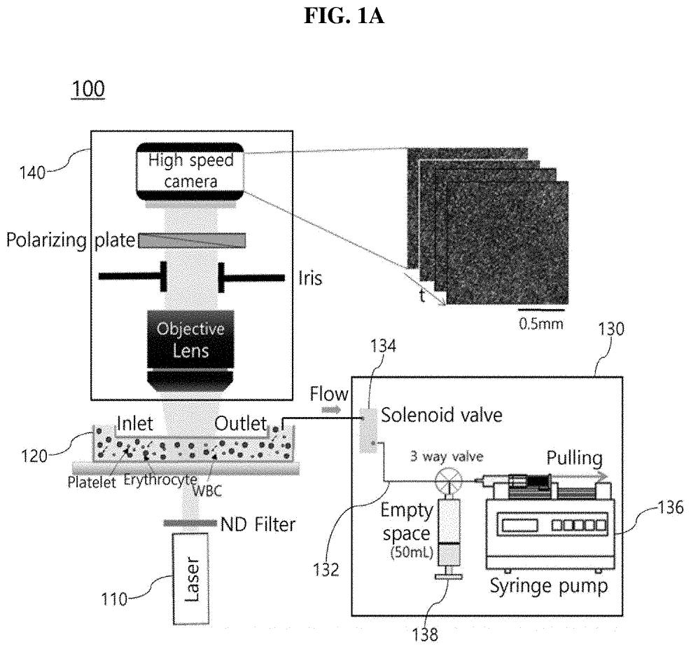

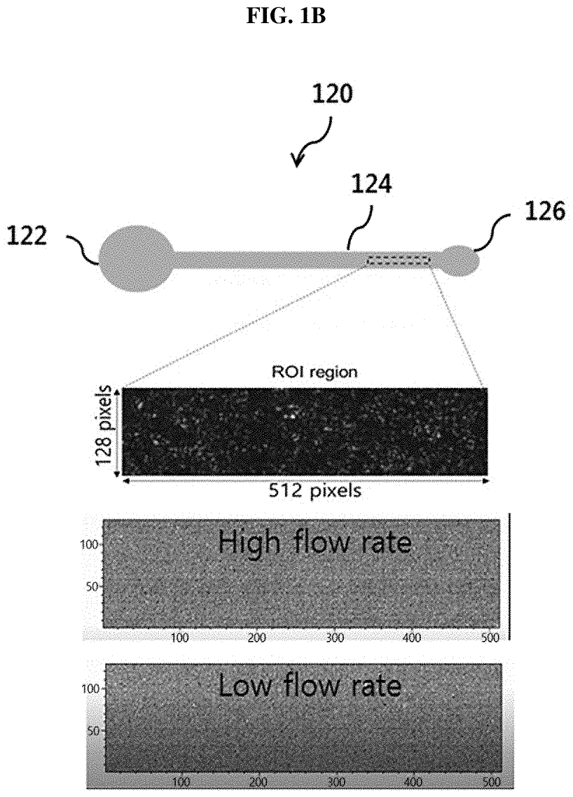

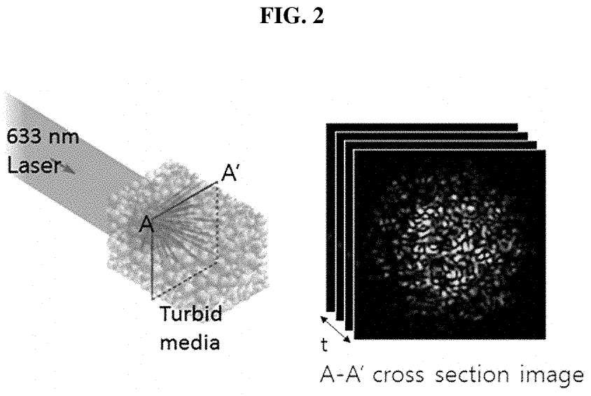

FIG. 1A is a view illustrating the functional configuration of an apparatus 100 for a platelet function test according to an exemplary embodiment of the present invention. FIG. 1B is a view illustrating a microfluidic chip 120 according to an exemplary embodiment of the present invention. FIG. 2 is a view illustrating a set of speckle images according to an exemplary embodiment of the present invention.

Referring to FIGS. 1A and 1B, an apparatus 100 for a platelet function test may include a laser unit 110, a microfluidic chip 120, a vacuum unit 130, an image capturing unit 140, and a control unit (not shown).

The laser unit 110 may irradiate a blood sample including platelets with a laser. In an exemplary embodiment, the microfluidic chip 120 may move the blood sample. In this case, the laser unit 110 may irradiate the blood sample moving through the microfluidic chip 120 with a laser.

In an exemplary embodiment, the microfluidic chip 120 may include an injection unit 122 into which a blood sample is injected, a movement channel 124 through which the injected blood sample moves, and a discharge unit 126 through which the moved blood sample is discharged.

The vacuum unit 130 may be coupled to the microfluidic chip 120 to move the blood sample by vacuum pressure.

In an exemplary embodiment, the vacuum unit 130 may include a movement tube 132 through which the discharged blood sample moves, a first valve 134 which is coupled to the movement tube 132 to open and close a flow of the blood sample, and a vacuum pump 136 which is coupled to the movement tube 132 to form a vacuum state inside the movement tube 132 according to the opening and closing of the first valve 134. For example, the first valve 134 may include a solenoid valve.

In an exemplary embodiment, the vacuum pump 136 may form a vacuum state inside the movement tube 132 after the first valve 134 is closed. For example, the vacuum pump 136 may include a syringe pump.

The first valve 134 may be opened after a vacuum state is formed inside the movement tube 132. Thus, the movement tube 132 may move a blood sample based on vacuum pressure generated as the first valve 134 is opened.

In an exemplary embodiment, the vacuum unit 130 may further include a buffer unit 138 which is coupled to the movement tube 132 to buffer the movement of the blood sample. In this case, the buffer unit 138 may be coupled to the movement tube 132 via a second valve. For example, the second valve may include a 3-way valve. The buffer unit 138 may include a vessel formed as an empty space.

The image capturing unit 140 may obtain a speckle image of the blood sample irradiated with the laser. In this case, the speckle image may include a speckle pattern which is irregularly generated by interference occurring when the laser is reflected by or passes through the blood sample.

In an exemplary embodiment, the image capturing unit 140 may include at least one of a lens onto which the laser emitted into the blood sample is projected, an iris, a polarizing plate and a camera.

The control unit may determine a speckle decorrelation time for platelets from the speckle image.

In an exemplary embodiment, the control unit may measure the function of platelets based on the speckle decorrelation time for platelets.

FIG. 2 is a view illustrating a set of speckle images according to an exemplary embodiment of the present invention.

Referring to FIG. 2, the speckle image may include a speckle pattern which is irregularly generated by interference occurring when the laser is reflected by or passes through the blood sample.

The speckle image may be obtained by the image capturing unit 140 at each specific cycle. In this case, the speckle decorrelation time may change the rate of deformation of the speckle pattern.

In an exemplary embodiment, the speckle decorrelation time may be determined based on the following <Equation 1>.

$${g}_{2} ( τ ) = \frac{< I ( {t}_{0} ) I ( {t}_{0} + τ ) >}{< I ( {t}_{0} ) > < I ( {t}_{0} + τ ) >} \qquad [ Equation 1 ]$$

Here, g₂(τ) indicates an intensity autocorrelation function, I(t₀) and I(t₀+τ) indicates an intensity at each time, and < > indicates the average calculation of all the captured data.

In an exemplary embodiment, <Equation 1> may be associated with a field autocorrelation function and may be represented by the following <Equation 2>.

g₂(τ)=1+β|g₁(τ)|² [Equation 2]

Here g₂(τ) indicates an intensity autocorrelation function, g₁(τ) indicates an electrical field autocorrelation function, and β indicates an experimental factor between 0 and 1, which is determined by the collection optics and the capture parameter (β=g₂(o)−1).

In this case, the autocorrelation function may be produced by an electrical signal g₁(τ) or a change in intensity g₂(τ).

In an exemplary embodiment, the electrical field autocorrelation function g₁(τ) may be represented by the following <Equation 3>.

$${g}_{1} ( τ ) = {\int }_{0}^{∞} P ( s ) exp [ ( - \frac{2 τ}{{τ}_{0}} ) \frac{s}{{l}^{*}} ] ds \qquad [ Equation 3 ]$$

Here, τ indicates decay time, τ₀=1/Dk₀², k₀(wavenumber)=2π/λ, D indicates the diffusion coefficient, l⁺ indicates the transport mean-free path, s indicates the path length, and P(s) indicates the distribution of the path length.

In an exemplary embodiment, the electrical field autocorrelations g₁(τ) may be represented by the following <Equation 4>.

$${g}_{1} ( τ ) = {\int }_{0}^{∞} P ( s ) exp [ - 2 [ τ / {τ}_{B} + {( τ / {τ}_{s} )}^{2} ] \frac{s}{{l}^{*}} ] ds \qquad [ Equation 4 ]$$

Here, τ_{B} indicates the relaxation rate from the Brownian motion, and τₛ indicates the relaxation rate from a laminar flow. That is, it can be confirmed that flow is not considered in <Equation 3>, and flow is considered in <Equation 4>.

$${g}_{1} ( τ ) = {\int }_{0}^{∞} exp [ \frac{τ}{η r} + {( \frac{Γ}{τ \sqrt{30}} )}^{2} ] ds \qquad [ Equation 5 ]$$

That is, it can be confirmed through <Equation 5> that the electrical field autocorrelation function g₁(τ) decays faster by the shear flow (Γ), the decrease in concentration (η) and the increase in particle size (r). In other words, the change in speckle decorrelation time may be determined by flow rate, concentration, and particle size.

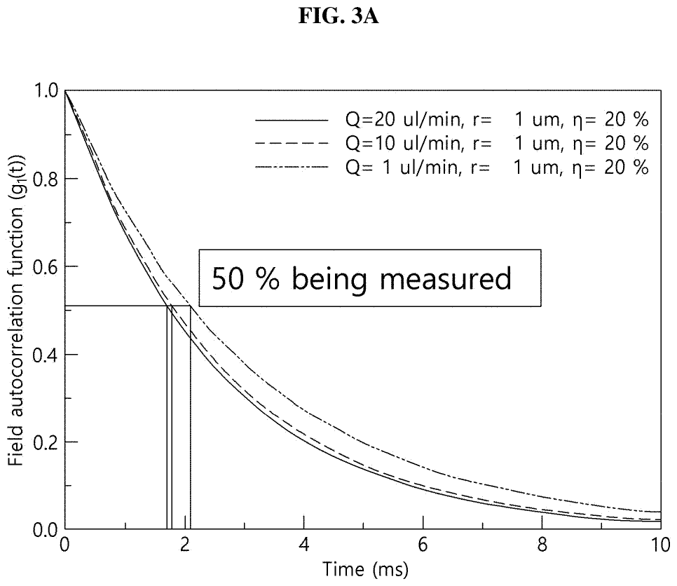

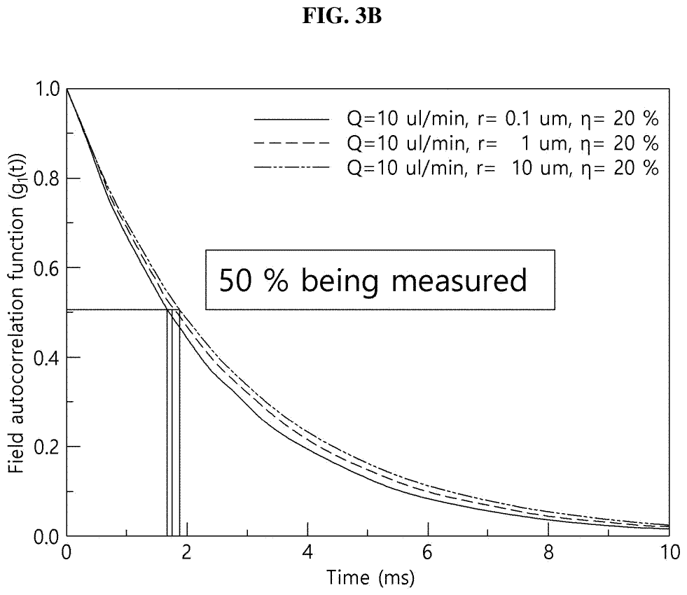

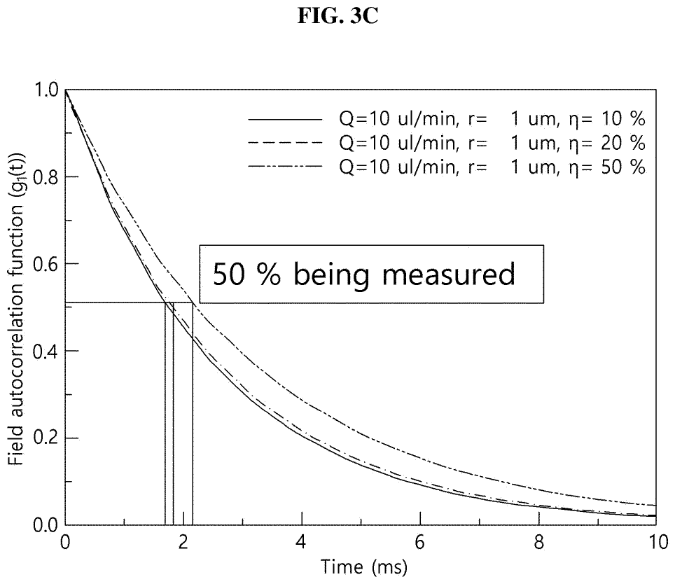

FIG. 3A is a view illustrating a speckle decorrelation time graph for the flow rate according to an exemplary embodiment of the present invention. FIG. 3B is a view illustrating a speckle decorrelation time graph for particle size according to an exemplary embodiment of the present invention. FIG. 3C is a view illustrating a speckle decorrelation time graph for particle concentration according to an exemplary embodiment of the present invention.

Referring to FIG. 3A, as a result of confirming the cases where the flow rate (Q) is 20 μl/min (1.83 ms), 10 μl/min (1.94 ms), and 1 μl/min (2.16 ms), it can be confirmed that as the flow rate is increased, the speckle decorrelation time is decreased.

Referring to FIG. 3B, as a result of confirming the cases where the particle size (r) is 0.1 μm (1.76 ms), 1 μm (1.83 ms), and 10 μm (1.91 ms), it can be confirmed that as the particle size is increased, the speckle decorrelation time is also increased.

Referring to FIG. 3C, as a result of confirming the cases where the concentration variation (η) is 10% (1.76 ms), 20% (1.89 ms), and 50% (2.19 ms), it can be confirmed that as the concentration variation is increased, the speckle decorrelation time is also increased.

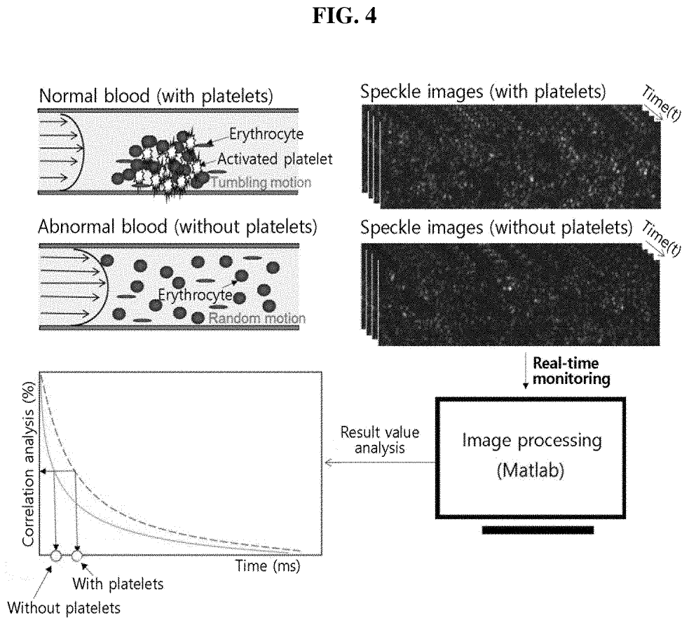

FIG. 4 is a view illustrating a speckle decorrelation time graph for the presence and absence of platelets according to an exemplary embodiment of the present invention.

Referring to FIG. 4, it can be confirmed that in the case of whole blood containing platelets in the movement tube 132, erythrocytes move in a tumbling motion. In contrast, it can be confirmed that in the case of platelet-poor blood in the movement tube 132, erythrocytes move in a random motion.

Thus, each speckle image for whole blood containing platelets and platelet-poor blood may be obtained at each time and analyzed in real time.

In this case, it can be confirmed that whole blood containing platelets has a longer speckle decorrelation time (τ) than platelet-poor blood, which is because there is a lower blood velocity due to platelet activation and also a slower change in speckle pattern.

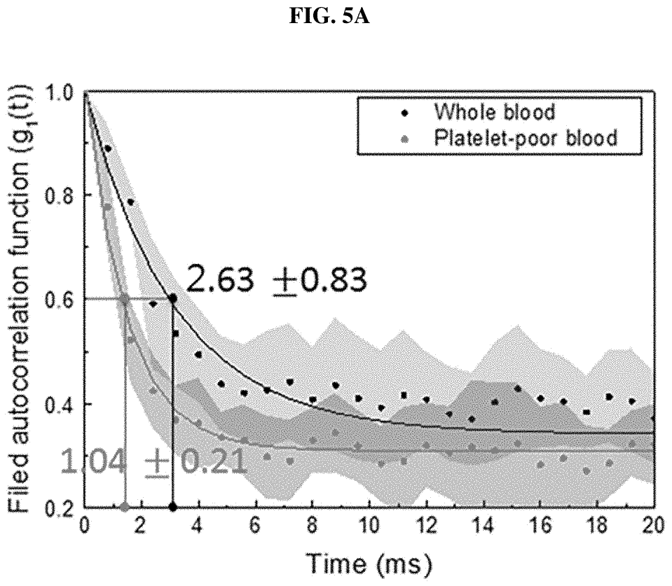

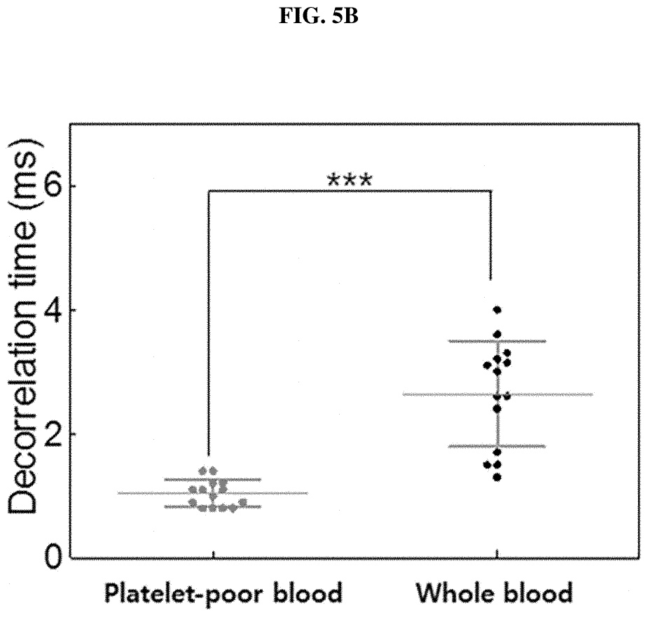

FIGS. 5A and 5B are views illustrating a speckle decorrelation time graph for the presence and absence of platelets according to an exemplary embodiment of the present invention.

Referring to FIGS. 5A and 5B, it can be confirmed that whole blood containing platelets has a longer speckle decorrelation time compared to platelet-poor blood.

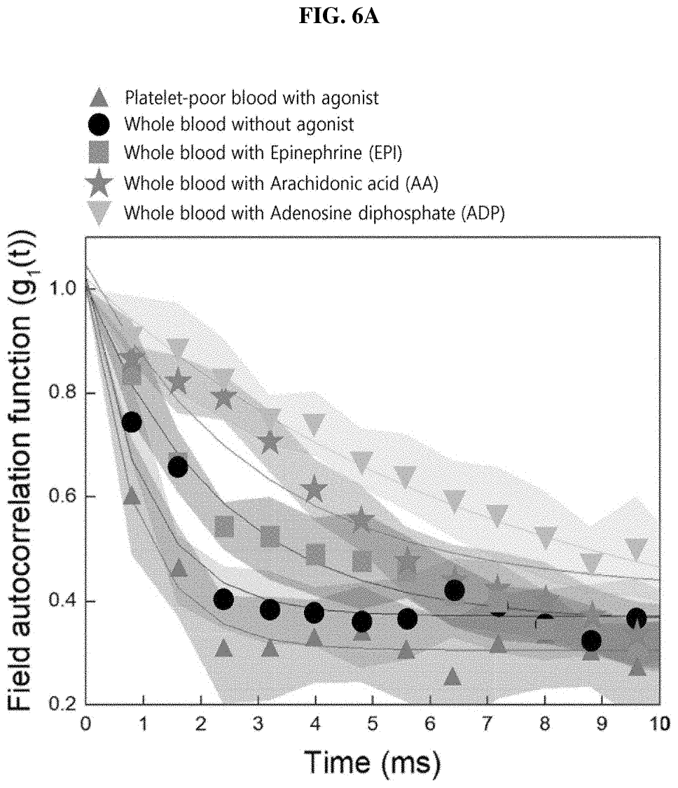

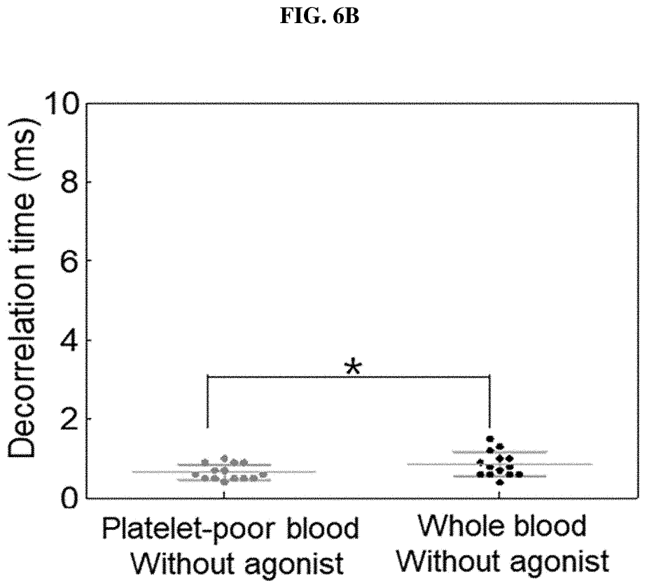

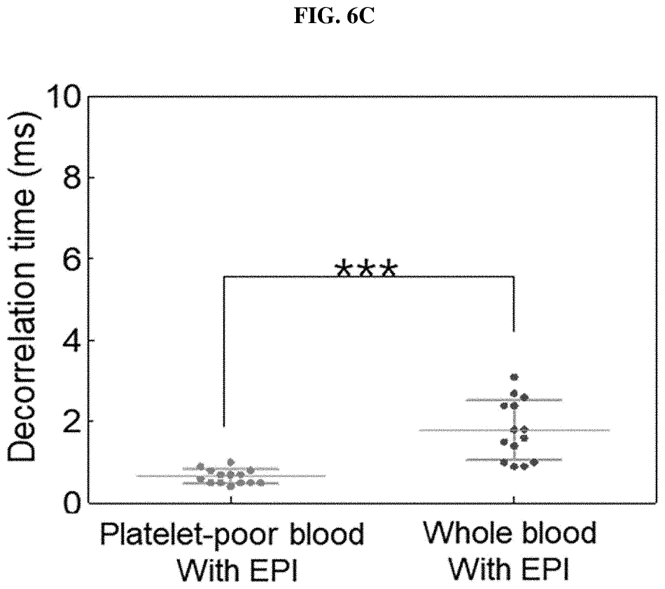

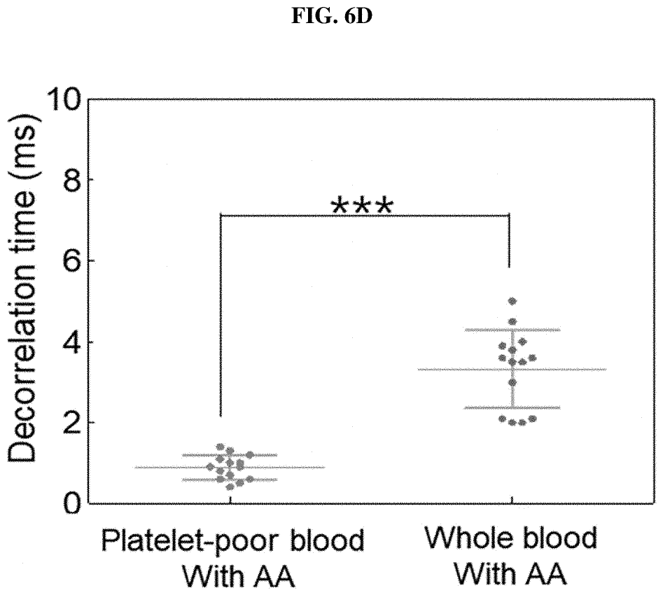

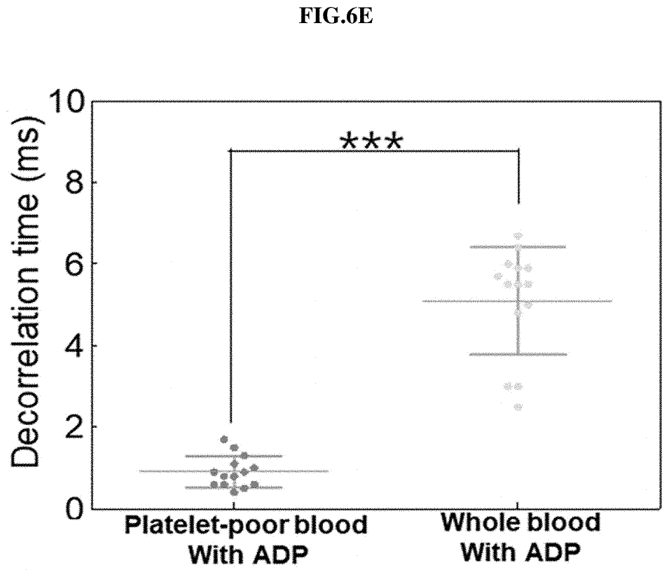

FIG. 6A to 6E are views illustrating a speckle decorrelation time graph for various platelet activation materials according to an exemplary embodiment of the present invention.

Referring to FIGS. 6A to 6E, a speckle decorrelation time test may be performed by utilizing a drug which activates platelets. In this case, it can be confirmed that the ADP platelet activator shows an approximate 5-fold or more difference between normal blood and abnormal blood. That is, the difference between normal and abnormal blood can be more clearly distinguished by utilizing a drug which activates platelets.

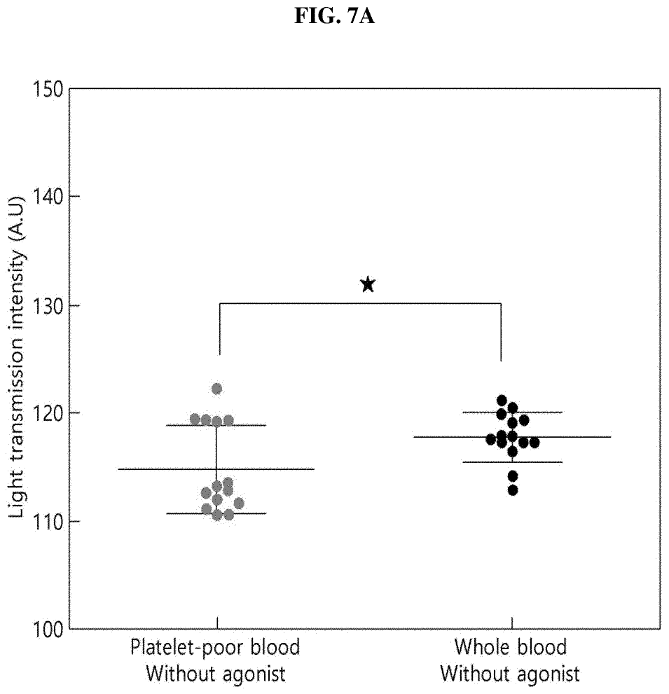

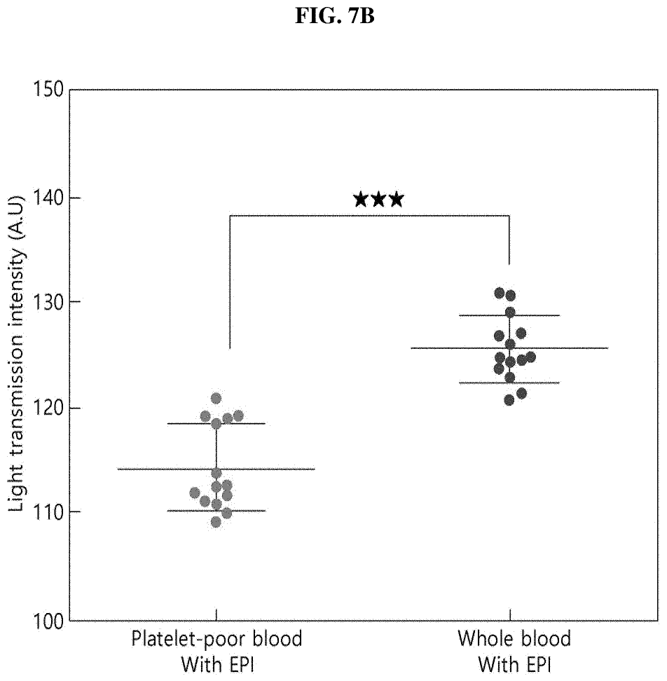

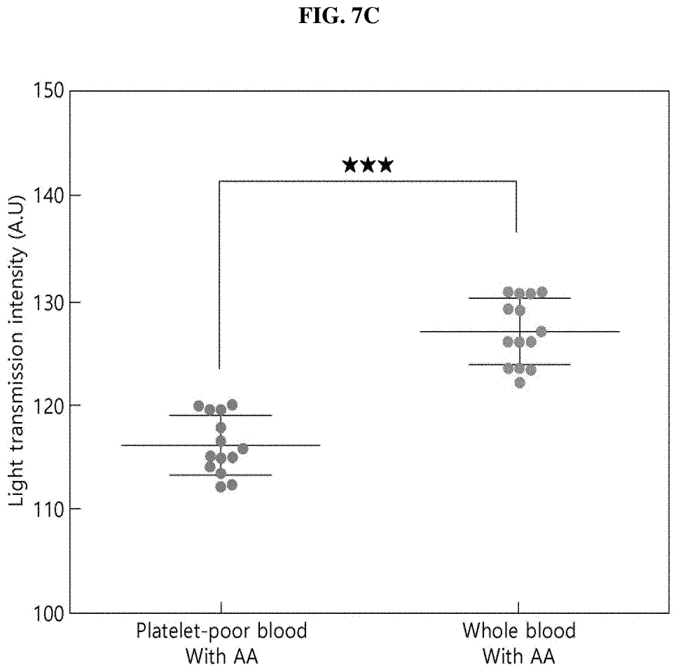

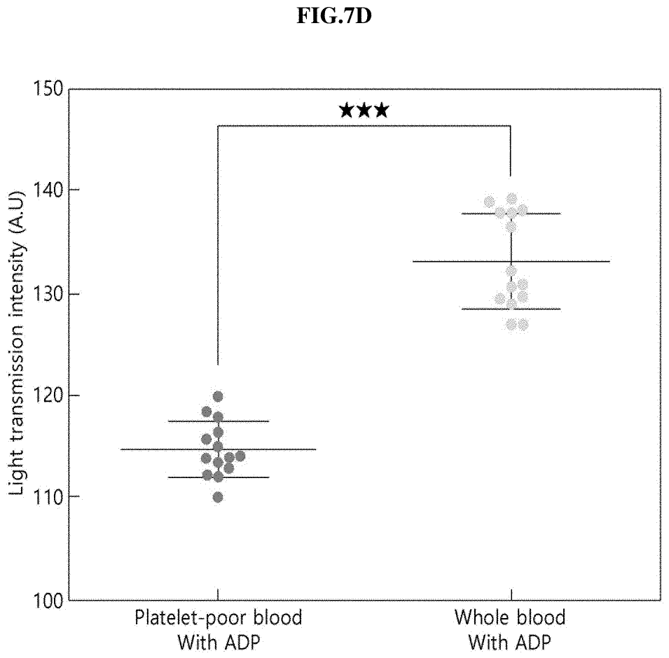

FIG. 7A to 7D are views illustrating a light transmittance graph according to an exemplary embodiment of the present invention.

Referring to FIGS. 7A to 7D, it can be confirmed that transmittance of light can also be measured, and a tendency similar to the speckle decorrelation time result is exhibited. Even in this case, the difference between normal and abnormal blood can be more clearly distinguished by utilizing the drug which activates platelets.

That is, according to various exemplary embodiments of the present invention, by collecting a small amount of blood and shortening a processing time, fast platelet diagnosis can be performed, antiplatelet drug resistance tests (for example, aspirin, clopidogrel) can be measured, so that synergistic effects can be obtained.

Further, according to various exemplary embodiments of the present invention, by analyzing the difference in the time when a speckle changes, the test time can be shortened using a system which has relatively high precision during the platelet test, has a low operation cost due to the configuration of a microfluidic chip and a simple system, and is capable of performing measurement in a short period of time. For example, the test may be performed in a required time of less than 1 minute using a blood volume of less than 10 μl.

According to an exemplary embodiment of the present invention, by analyzing the difference in the time when a speckle changes, the test time can be shortened using a system which has relatively high precision during the platelet test, has a low operation cost due to the configuration of a microfluidic chip and a simple system, and is capable of performing measurement in a short period of time.

The effects of the present invention are not limited to the above-described effects, and the potential effects expected by the technical features of the present invention will be clearly understood from the following description.

The above description merely illustrates the technical spirit of the present invention, and those skilled in the art can make various changes and modifications without departing from the scope of essential characteristics of the present invention.

Accordingly, the embodiments disclosed in the present invention are intended not to limit but to describe the technical spirit of the present invention, and the scope of the technical spirit of the present invention is not limited by these embodiments.

The scope of protection of the present invention must be interpreted by the accompanying claims, and it should be interpreted that all the technical spirit within a scope equivalent thereto are included in the scope of rights of the present invention.

What is claimed is:

1. An apparatus for a platelet function test, comprising:
a laser unit for irradiating a blood sample comprising platelets with a laser;
an image capturing unit for obtaining a speckle image of the blood sample irradiated with the laser; and
a control unit for determining the speckle decorrelation time for platelets.

2. The apparatus of claim 1, further comprising a microfluidic chip for moving the blood sample,
wherein the laser unit irradiates the blood sample moving through the microfluidic chip with a laser.

3. The apparatus of claim 2, wherein the microfluidic chip comprises
an injection unit into which the blood sample is injected,
a movement channel through which the injected blood sample moves, and
a discharge unit through which the moved blood sample is discharged.

4. The apparatus of claim 3, further comprising a vacuum unit which is coupled to the microfluidic chip to move the blood sample by vacuum pressure.

5. The apparatus of claim 4, wherein the vacuum unit comprises
a movement tube through which the discharged blood sample moves,
a first valve which is coupled to the movement tube to open and close a flow of the blood sample, and
a vacuum pump which is coupled to the movement tube to form a vacuum state inside the movement tube according to the opening and closing of the first valve.

6. The apparatus of claim 5, wherein the vacuum unit further comprises a buffer unit which is coupled to the movement tube to buffer the movement of the blood sample.

7. The apparatus of claim 5, wherein the vacuum pump forms a vacuum state inside the movement tube after the first valve is closed,
the first valve is opened after the vacuum state inside the movement tube is formed, and
the movement tube moves the blood sample based on vacuum pressure generated as the first valve is opened.

8. The apparatus of claim 1, wherein the control unit measures the function of platelets based on the speckle decorrelation time for platelets.
